(19) United States
(12) Patent Application Publication
(10) Pub. No.: US 2023/0290520 A1
(43) Pub. Date: Sep. 14, 2023

(54) METHOD AND ELECTRONIC DEVICE OF CHECKING DRUG INTERACTION

(71) Applicants: Acer Incorporated, New Taipei City (TW); National Yang Ming Chiao Tung University, Hsinchu (TW)

(72) Inventors: Pei-Jung Chen, New Taipei City (TW); Tsung-Hsien Tsai, New Taipei City (TW); Liang-Kung Chen, Hsinchu (TW); Fei-Yuan Hsiao, Hsinchu (TW); Shih-Tsung Huang, Hsinchu (TW)

(73) Assignees: Acer Incorporated, New Taipei City (TW); National Yang Ming Chiao Tung University, Hsinchu (TW)

(21) Appl. No.: 17/842,809

(22) Filed: Jun. 17, 2022

(30) Foreign Application Priority Data
Mar. 10, 2022 (TW) 111108870

(51) Int. Cl. G16H 70/40; G16H 10/60

(52) U.S. Cl. CPC G16H 70/40; G16H 10/60

(57) ABSTRACT

A method and an electronic device of checking drug interaction are provided. The method includes: generating a first odds ratio (OR) between a first drug combination and a hospitalization event, a second OR between a second drug combination and the hospitalization event, and a third OR between a third drug combination and the hospitalization event according to a plurality of medical records; generating a first fraction corresponding to a first drug according to the second OR; generating a second fraction corresponding to a second drug according to the third OR; and outputting the first drug combination in response to the first OR being greater than a first threshold, a sum of the first fraction and the second fraction being greater than a second threshold, and a quotient of the first fraction and the second fraction being less than a third threshold.

### CROSS-REFERENCE TO RELATED APPLICATION

This application claims the priority benefit of Taiwan application serial no. 111108870, filed on Mar. 10, 2022. The entirety of the above-mentioned patent application is hereby incorporated by reference herein and made a part of this specification.

### BACKGROUND

### Technical Field

The disclosure relates to a method and an electronic device of checking drug interaction.

### Description of Related Art

Patients often need to take multiple medications in a period of time. Interaction between drugs may lead to serious adverse reaction and cause the patients to be hospitalized unexpectedly. In order to avoid such a situation, it is necessary to check the interaction between various drug combinations. However, considering the huge number of drug combinations, it may be inefficient to check the drug combinations one by one. Therefore, how to quickly find out a high-risk drug combination is an issue that needs to be addressed in this field.

### SUMMARY

The disclosure provides a method and an electronic device of checking drug interaction, which output a drug combination with a high risk for the user's reference.

A method of checking drug interaction according to the disclosure includes the following. A plurality of medical records are obtained, and at least one of the plurality of medical records indicates whether a patient taking a first drug combination has a hospitalization event. A drug combination set is generated according to the plurality of medical records. The drug combination set includes the first drug combination, a second drug combination, and a third drug combination. The first drug combination and the second drug combination both include a first drug, and the first drug combination and the third drug combination both include a second drug. A first odds ratio between the first drug combination and the hospitalization event, a second odds ratio between the second drug combination and the hospitalization event, and a third odds ratio between the third drug combination and the hospitalization event are generated according to the plurality of medical records. A first fraction corresponding to the first drug is generated according to the second odds ratio, and the first fraction is negatively correlated with the second odds ratio. A second fraction corresponding to the second drug is generated according to the third odds ratio, and the second fraction is negatively correlated with the third odds ratio, and the first fraction is greater than or equal to the second fraction. The first drug combination is output in response to the first odds ratio being greater than a first threshold, a sum of the first fraction and the second fraction being greater than a second threshold, and a quotient of the first fraction and the second fraction being less than a third threshold.

In an embodiment of the disclosure, generating the first fraction corresponding to the first drug according to the second odds ratio includes the following. The second drug combination is marked in response to the second odds ratio being greater than a risk threshold. A third fraction is generated according to the second drug combination marked. The third fraction is equal to the number of drug combinations including the first drug but not including the second drug and being marked in the drug combination set, divided by the number of drug combinations including the first drug but not including the second drug in the drug combination set. The first fraction is calculated according to the third fraction, and a sum of the first fraction and the third fraction is equal to 1.

In an embodiment of the disclosure, generating the drug combination set according to the plurality of medical records includes the following. A screening process is executed to generate a first unique drug combination set, including: generating K topic vectors including a first topic vector according to the plurality of medical records and a latent Dirichlet allocation model, K being a first topic number, the K topic vectors respectively corresponding to K topics, the K topics including a first topic corresponding to the first topic vector, and the first topic vector including a probability distribution of all drug combinations; selecting a plurality of important drug combinations, starting with a drug combination with a maximum probability, from the first topic vector to generate a first important drug combination set; and determining the first unique drug combination set according to the first important drug combination set. The drug combination set is generated according to the first unique drug combination set.

In an embodiment of the disclosure, the K topics include a second topic, and determining the first unique drug combination set according to the first important drug combination set includes the following. A first important drug combination is deleted from the first important drug combination set to generate the first unique drug combination set in response to the first important drug combination being included in the first important drug combination set corresponding to the first topic and a second important drug combination set corresponding to the second topic.

In an embodiment of the disclosure, generating the drug combination set according to the first unique drug combination set includes the following. The screening process is repeatedly executed multiple times to generate a plurality of unique drug combination sets including the first unique drug combination set. A first stable drug combination set corresponding to the first topic is generated according to the first drug combination in response to the number of the first drug combinations in the plurality of unique drug combination sets being greater than a number threshold. The drug combination set is generated according to the first stable drug combination set.

In an embodiment of the disclosure, generating the drug combination set according to the first stable drug combination set includes the following. A plurality of medical record vectors respectively corresponding to the plurality of medical records are generated according to the plurality of medical records and the latent Dirichlet allocation model. Each of the plurality of medical record vectors includes a probability distribution of the K topics. A medical record set corresponding to the first topic among the plurality of medical records is determined according to the probability distribution of the K topics. A ratio of at least one medical record in the medical record set to the medical record set is calculated, and the at least one medical record indicates at least one drug combination in the first stable drug combination set. The drug combination set is generated according to the first stable drug combination set in response to the ratio being greater than a ratio threshold, and the drug combination set includes a plurality of drug combinations in the first stable drug combination set.

In an embodiment of the disclosure, a first medical record in the medical record set corresponds to a first probability distribution of the K topics, and determining the medical record set corresponding to the first topic among the plurality of medical records according to the probability distribution of the K topics includes the following. It is determined that the first medical record corresponds to the first topic in response to a maximum probability in the first probability distribution corresponding to the first topic.

In an embodiment of the disclosure, the method of checking drug interaction further includes the following. A first index corresponding to the first topic number and a second index corresponding to a second topic number are generated according to the plurality of medical records and the latent Dirichlet allocation model. The first index and the second index are compared to select the first topic number from the first topic number and the second topic number as K.

In an embodiment of the disclosure, generating the first index corresponding to the first topic number includes the following. The K topic vectors are generated according to the plurality of medical records, the latent Dirichlet allocation model, and the first topic number. An average similarity of all 2-combinations of the K topic vectors is calculated as the first index.

In an embodiment of the disclosure, generating the first index corresponding to the first topic number includes the following. A plurality of medical record vectors respectively corresponding to the plurality of medical records are generated according to the plurality of medical records, the latent Dirichlet allocation model, and the first topic number. Each of the plurality of medical record vectors includes a probability distribution of the K topics. At least one medical record corresponding to the first topic among the plurality of medical records is determined according to the probability distribution of the K topics. A ratio is calculated according to the number of the at least one medical record and a total number of the plurality of medical records as the first index.

In an embodiment of the disclosure, determining the at least one medical record corresponding to the first topic among the plurality of medical records according to the probability distribution of the K topics includes the following. A first probability distribution corresponding to the K topics of the at least one medical record is obtained from the plurality of medical record vectors. It is determined that the at least one medical record corresponds to the first topic in response to a maximum probability in the first probability distribution corresponding to the first topic and being greater than a probability threshold.

In an embodiment of the disclosure, generating the first index corresponding to the first topic number includes the following. A plurality of medical record vectors respectively corresponding to the plurality of medical records are generated according to the plurality of medical records, the latent Dirichlet allocation model, and the first topic number. Each of the plurality of medical record vectors includes a probability distribution of the K topics. The plurality of medical records are divided into K groups according to the probability distribution of the K topics, and the K groups respectively correspond to the K topics. A first statistical value of inter-group distances is calculated according to the K groups. A second statistical value of intra-group distances is calculated according to the K groups. A ratio of the first statistical value to the second statistical value is calculated as the first index.

In an embodiment of the disclosure, calculating the first statistical value of the inter-group distances according to the K groups includes the following. A plurality of distances between the K topic vectors are calculated. The first statistical value is obtained by adding the plurality of distances.

In an embodiment of the disclosure, the K groups include a first group and a second group, and calculating the second statistical value of the intra-group distances according to the K groups includes the following. A plurality of distances between a plurality of elements in the first group are calculated to generate a first sum of intra-group distances corresponding to the first group. The second statistical value is obtained by adding the sum of the first intra-group distances corresponding to the first group and a second sum of intra-group distances corresponding to the second group.

An electronic device of checking drug interaction according to the disclosure includes a processor and a transceiver. The processor is coupled to the transceiver and configured to: obtain, through the transceiver, a plurality of medical records, at least one of the plurality of medical records indicating whether a patient taking a first drug combination has a hospitalization event; generate a drug combination set according to the plurality of medical records, the drug combination set including the first drug combination, a second drug combination, and a third drug combination, the first drug combination and the second drug combination both including a first drug, and the first drug combination and the third drug combination both including a second drug; generate a first odds ratio between the first drug combination and the hospitalization event, a second odds ratio between the second drug combination and the hospitalization event, and a third odds ratio between the third drug combination and the hospitalization event according to the plurality of medical records; generate a first fraction corresponding to the first drug according to the second odds ratio, the first fraction being negatively correlated with the second odds ratio; generate a second fraction corresponding to the second drug according to the third odds ratio, the second fraction being negatively correlated with the third odds ratio, and the first fraction being greater than or equal to the second fraction; and output, through the transceiver, the first drug combination in response to the first odds ratio being greater than a first threshold, a sum of the first fraction and the second fraction being greater than a second threshold, and a quotient of the first fraction and the second fraction being less than a third threshold.

Based on the above, at least one embodiment of the disclosure can find out a drug combination with a high risk from numerous drug combinations, and can confirm that the high risk of the drug combination does not result from the drugs themselves in the drug combination but from the interaction between the drugs.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic diagram showing an electronic device of checking drug interaction according to an embodiment of the disclosure.

FIG. 2 is a schematic diagram showing the relationship between the average similarity between topics and the topic number K according to an embodiment of the disclosure.

FIG. 3 is a schematic diagram showing the relationship between the ratio of medical records with a biased topic and the topic number K according to an embodiment of the disclosure.

FIG. 4 is a schematic diagram showing the relationship between the clustering efficiency index and the topic number K according to an embodiment of the disclosure.

FIG. 5 is a schematic diagram showing finding important drug combinations using an elbow method according to an embodiment of the disclosure.

FIG. 6 is a flowchart showing a method of checking drug interaction according to an embodiment of the disclosure.

### DETAILED DESCRIPTION OF DISCLOSED EMBODIMENTS

In order to make the disclosure more comprehensible, the following specific embodiments are given as examples showing that the disclosure can indeed be implemented. Additionally, where possible, elements/components/steps using the same reference numerals in the drawings and embodiments represent the same or similar parts.

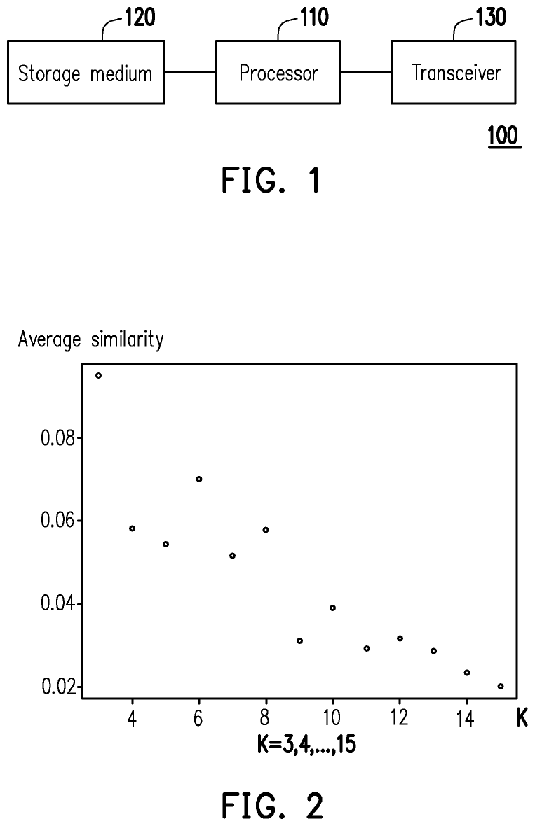

FIG. 1 is a schematic diagram of an electronic device 100 of checking drug interaction according to an embodiment of the disclosure. The electronic device 100 includes a processor 110, a storage medium 120, and a transceiver 130.

The processor 110 is, for example, a central processing unit (CPU), a programmable general-purpose or special-purpose micro control unit (MCU), a microprocessor, a digital signal processor (DSP), a programmable controller, an application specific integrated circuit (ASIC), a graphics processing unit (GPU), an image signal processor (ISP), an image processing unit (IPU), an arithmetic logic unit (ALU), a complex programmable logic device (CPLD), a field programmable gate array (FPGA), other similar elements or a combination of the foregoing elements. The processor 110 may be coupled to the storage medium 120 and the transceiver 130, and access and execute a plurality of modules and various application programs stored in the storage medium 120.

The storage medium 120 is, for example, any type of fixed or removable random access memory (RAM), read-only memory (ROM), flash memory, hard disk drive (HDD), solid state drive (SSD), other similar elements or a combination of the foregoing elements. The storage medium 120 is configured to store a plurality of modules or various application programs that can be executed by the processor 110.

The transceiver 130 is configured to transmit and receive signals in a wireless or wired manner. The transceiver 130 may also execute operations such as low noise amplification, impedance matching, frequency mixing, up or down frequency conversion, filtering, amplification, and the like.

The processor 110 may obtain N medical records respectively corresponding to N patients through the transceiver 130, wherein N is a positive integer. The medical record may indicate a drug combination that the patient has taken, wherein one drug combination may include two drugs. For example, a drug combination (α, β) may include drug α and drug β. The medical record may also indicate whether the patient has had an unexpected hospitalization event. Table 1 is a schematic diagram of N medical records. Taking medical record #1 as an example, medical record #1 indicates that the patient corresponding to medical record #1 has taken a combination (A, B) of drug A and drug B, a combination (X, Y) of drug X and drug Y, and a combination (U, W) of drug U and drug W. Medical record #1 also indicates that the patient corresponding to medical record #1 has had an unexpected hospitalization event. That is, the drug combination that the patient has taken may lead to the unexpected hospitalization event. Taking medical record #N as an example, medical record #N indicates that the patient corresponding to medical record #N has taken a combination (C, D) of drug C and drug D and a combination (O, P) of drug O and drug P. Medical record #N also indicates that the patient corresponding to medical record #N has not had an unexpected hospitalization event. That is, the drug combination that the patient had taken did not lead to an unexpected hospitalization event. The drug combination on the medical record is recorded in the form of an anatomical therapeutic chemical (ATC) code, for example.

TABLE 1

| Medical | | Hospitalization |
|---|---|---|
| record | Drug combination | event |
| #1 | (A, B), (X, Y), (U, W) | Yes |
| #2 | (G, H) | Yes |
| . . . | . . . | . . . |
| #N | (C, D), (O, P) | Yes |

In this embodiment, it is assumed that N medical records record M drug combinations in total, wherein M is a positive integer. Taking Table 1 as an example, the M drug combinations at least include drug combinations (A, B), (X, Y), (U, W), (G, H), (C, D), and (O, P). The M drug combinations are the union of all the drug combinations in the N medical records.

The processor 110 may generate a drug combination set including a plurality of drug combinations according to the N medical records, and then select a drug combination with a high risk of interaction from the drug combination set for the user's reference. First, the processor 110 may analyze the N medical records using a latent Dirichlet allocation (LDA) model. The parameters of the LDA model may include a topic number K, wherein K is a positive integer. K may determine that the output generated by the LDA model is related to K topics. The processor 110 may first determine the value of the optimal topic number Kopt.

Specifically, the output of the LDA model can be associated with topics and words. The processor 110 may input the N medical records to the LDA model to generate K topic vectors respectively corresponding to the K topics (or referred to as drug patterns). Each topic vector may include a probability distribution of all the drug combinations (that is, M drug combinations). The drug combination is the word of the LDA model. The topic vector includes the probability distribution of all the words (word distribution). In other words, the topic vector may be a vector including M probabilities, wherein the M probabilities respectively correspond to the M drug combinations.

Table 2 is an example of the K topic vectors. Taking the topic vector corresponding to topic #1 as an example, the topic vector may include at least the probability value “0.20” corresponding to the drug combination (A, B), the probability value “0.05” corresponding to the drug combination (C, D), and the probability value “0.20” corresponding to the drug combination (X, Y). The sum of all elements (that is, M probabilities) in the topic vector is equal to “1.”

TABLE 2

| | Drug | | | | |
|---|---|---|---|---|---|
| Number | combination | Topic #1 | Topic #2 | . . . | Topic #K |
| #1 | (A, B) | 0.20 | 0.00 | . . . | 0.10 |
| #2 | (C, D) | 0.05 | 0.10 | . . . | 0.20 |
| . . . | . . . | . . . | . . . | . . . | . . . |
| #M | (X, Y) | 0.20 | 0.15 | . . . | 0.05 |

Further, the LDA model may also generate N medical record vectors corresponding to the N medical records respectively according to the N medical records. Each medical record vector may include a probability distribution of K topics (topic distribution). In other words, the medical record vector may be a vector including K probabilities, wherein the K probabilities respectively correspond to the K topics. Table 3 is an example of N medical record vectors. Taking the medical record vector corresponding to medical record #1 as an example, the medical record vector may include at least the probability value “0.20” corresponding to topic #1, the probability value “0.00” corresponding to topic #2, and the probability value “0.10” corresponding to topic #K. The sum of all elements (that is, K probabilities) in the medical record vector is equal to “1.”

TABLE 3

| Medical record | Topic #1 | Topic #2 | . . . | Topic #K |
|---|---|---|---|---|
| #1 | 0.20 | 0.00 | . . . | 0.10 |
| #2 | 0.05 | 0.10 | . . . | 0.20 |
| . . . | . . . | . . . | . . . | . . . |
| #N | 0.00 | 0.05 | . . . | 0.15 |

The processor 110 may determine the value of the optimal topic number Kopt according to factors such as the similarity between topics, the ratio of medical records with a biased topic (that is, with a biased drug pattern), and the clustering efficiency index.

In order to find out various drug patterns, the greater the difference between the topics, the better. That is, the lower the similarity between the topics, the better. In an embodiment, the processor 110 may calculate the average similarity of all 2-combinations (C_{K}² in total) of the K topic vectors as an index for determining the optimal topic number Kopt. The similarity is, for example, cosine similarity or Jaccard similarity, but the disclosure is not limited thereto. Taking Table 2 as an example, it is assumed that K is equal to “3.” The processor 110 may calculate the similarity between the topic vector [0.20 0.05 . . . 0.20] corresponding to topic #1 and the topic vector [0.00 0.10 . . . 0.15] corresponding to topic #2, the similarity between the topic vector [0.20 0.05 . . . 0.20] corresponding to topic #1 and the topic vector [0.10 0.20 . . . 0.05] corresponding to topic #3, and the similarity between the topic vector [0.00 0.10 . . . 0.15] corresponding to topic #2 and the topic vector [0.10 0.20 . . . 0.05] corresponding to topic #3, and calculate the average of the three similarities to obtain the average similarity, as shown in Table 4.

TABLE 4

| Topic | Similarity |
|---|---|
| (#1, #2) | 0.82 |
| (#1, #3) | 0.58 |
| (#2, #3) | 0.68 |

Average similarity: 0.69

FIG. 2 is a schematic diagram showing the relationship between the average similarity between topics and the topic number K according to an embodiment of the disclosure. After comparing the average similarity corresponding to each topic number K, it can be found that the average similarity between topics decreases as the value K increases. Therefore, if the average similarity is used as an index for determining the optimal topic number Kopt, the processor 110 may select a greater value as the optimal topic number Kopt.

In order for each distinct medical record (or patient) to be classified in a representative drug pattern, the higher the ratio of medical records with a biased topic, the better. In an embodiment, the processor 110 may calculate a ratio according to the number of medical records corresponding to a specific topic and the total number of all medical records (that is, N) as an index for determining the optimal topic number Kopt. Specifically, the processor 110 may determine whether the medical record vector is biased to a specific topic according to the probability distribution of the K topics in the medical record vector. If the maximum probability in the probability distribution corresponds to a specific topic and the maximum probability is greater than a probability threshold, the processor 110 may determine that the medical record vector (or medical records) is biased to the specific topic.

TABLE 5

| | Medical | Medical | Medical | Medical | Medical |
|---|---|---|---|---|---|
| Topic | record #1 | record #2 | record #3 | record #4 | record #5 |
| #1 | 0.60 | 0.05 | 0.40 | 0.10 | 0.35 |
| #2 | 0.30 | 0.70 | 0.25 | 0.20 | 0.30 |
| #3 | 0.10 | 0.25 | 0.35 | 0.70 | 0.35 |

Table 5 is an example of a plurality of medical record vectors, wherein it is assumed that N is equal to “5,” K is equal to “3,” and the probability threshold is equal to “0.50.” Taking medical record #1 as an example, the processor 110 may determine that medical record #1 is biased to topic #1 in response to the maximum probability “0.60” in medical record #1 corresponding to topic #1 and being greater than the probability threshold “0.50.” Taking medical record #3 as an example, the processor 110 may determine that medical record #3 is not biased to any topic in response to the maximum probability “0.40” in medical record #3 is less than or equal to “0.50.” Accordingly, the processor 110 may obtain the topic to which each of all the medical records is biased according to the data in Table 5, as shown in Table 6.

TABLE 6

| | Medical | Medical | Medical | Medical | Medical |
|---|---|---|---|---|---|
| | record #1 | record #2 | record #3 | record #4 | record #5 |
| Biased topic | Topic #1 | Topic #2 | N/A | Topic #3 | N/A |

After obtaining the topic to which each medical record is biased, the processor 110 may calculate the ratio of the number of medical records corresponding to a specific topic to the total number of all medical records as an index. Taking Table 6 as an example, the number of medical records (that is, medical record #1, medical record #2, and medical record #4) corresponding to a specific topic is equal to “3” and the total number N of all medical records is equal to “5.” The processor 110 may calculate the ratio “3/5” as an index for determining the optimal topic number Kopt.

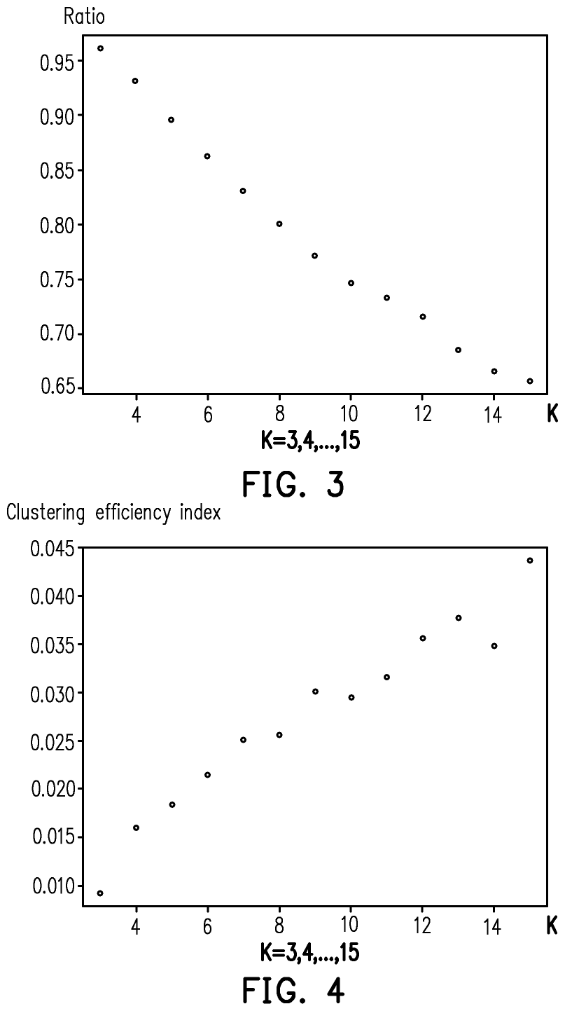

FIG. 3 is a schematic diagram showing the relationship between the ratio of medical records with a biased topic and the topic number K according to an embodiment of the disclosure. After comparing the ratio corresponding to each topic number K, it can be found that the ratio of medical records with a biased topic increases as the value K decreases. Therefore, if the ratio of medical records with a biased topic is used as an index for determining the optimal topic number Kopt, the processor 110 may select a smaller value as the optimal topic number Kopt.

In an embodiment, the processor 110 may use the clustering efficiency index as an index for determining the optimal topic number Kopt. First, the processor 110 may assign medical records to groups of specific topics. Specifically, the processor 110 may assign the medical records to one of K groups according to the probability distribution corresponding to the K topics in the medical record vector of the medical records, wherein the K groups respectively correspond to the K topics. The processor 110 may, for example, assign the medical records to the group corresponding to the topic with the maximum probability in the medical record vector. Taking Table 5 as an example, the processor 110 may assign medical record #1 to the group corresponding to topic #1, assign medical record #2 to the group corresponding to topic #2, assign medical record #3 to the group corresponding to topic #1, and assign medical record #4 to the group corresponding to topic #3. If there are multiple maximum probabilities in the medical record vector, the processor 110 may assign the medical records to the topic corresponding to one of the multiple maximum probabilities according to a preset rule or randomly. Taking Table 5 as an example, the processor 110 may assign medical record #5 to the group corresponding to one of topic #1 and topic #3 according to a preset rule or randomly.

After assigning N medical records to the groups, the processor 110 may calculate a first statistical value corresponding to the inter-group distances and a second statistical value corresponding to the intra-group distances according to the K groups. The clustering efficiency index may be equal to the ratio of the first statistical value to the second statistical value.

The first statistical value is, for example, the sum of a plurality of distances between K topic vectors. Specifically, the first statistical value may be the sum of the distances of all 2-combinations of the K groups. For example, it is assumed that K is equal to “3” and the K groups include group #1, group #2, and group #3. The processor 110 may calculate the distance between group #1 and group #2, the distance between group #1 and group #3, and the distance between group #2 and group #3, and calculate the sum of the three distances to obtain the first statistical value. The distance may be calculated according to the distance between topic vectors. For example, the distance between group #1 corresponding to topic #1 and group #2 corresponding to topic #2 may be equal to the distance between the topic vector (for example, [0.20 0.05 . . . 0.20] in Table 2) corresponding to topic #1 and the topic vector (for example, [0.00 0.10 . . . 0.15] in Table 2) corresponding to topic #2. The greater the distance between the groups, the better the efficiency of clustering. Therefore, the first statistical value may be proportional to the clustering efficiency index.

The second statistical value is, for example, the sum of K intra-group distances respectively corresponding to the K groups. The intra-group distance of each group may be equal to the sum of a plurality of distances between a plurality of elements in the group. In more detail, the intra-group distance corresponding to a group may be the sum of the distances of all 2-combinations of all elements in the group. Taking group #1 corresponding to topic #1 as an example, it is assumed that group #1 includes three elements such as medical record #1, medical record #2, and medical record #3 (that is, three of the N medical records correspond to topic #1). The processor 110 may calculate the distance between medical record #1 and medical record #2, the distance between medical record #1 and medical record #3, and the distance between medical record #2 and medical record #3, and add the three distances to obtain the intra-group distance of group #1.

In an embodiment, the processor 110 may vectorize the drug combinations recorded in the medical records to calculate the distance between the medical records. Taking medical record #1 and medical record #2 in Table 1 as an example, to calculate the distance between medical record #1 and medical record #2, the processor 110 may convert the drug combination “(A, B), (X, Y), (U, W)” of medical record #1 into a vector and convert the drug combination “(G, H)” of medical record #2 into another vector. The processor 110 may calculate the distance between the two vectors as the distance between medical record #1 and medical record #2. The shorter the distance between the elements in the group, the better the efficiency of clustering. Therefore, the second statistical value may be inversely proportional to the clustering efficiency index.

FIG. 4 is a schematic diagram showing the relationship between the clustering efficiency index and the topic number K according to an embodiment of the disclosure. After comparing the ratio corresponding to each topic number K, it can be found that the clustering efficiency index increases as the value K increases. Therefore, if the clustering efficiency index is used as an index for determining the optimal topic number Kopt, the processor 110 may select a greater value as the optimal topic number Kopt.

The processor 110 may determine the optimal topic number Kopt according to FIG. 2, FIG. 3, and FIG. 4. After determining the optimal topic number Kopt, the processor 110 may set the optimal topic number Kopt as the topic number K in the parameters of the LDA model. Then, the processor 110 may generate K topic vectors respectively corresponding to K topics (as shown in Table 2) and N medical record vectors respectively corresponding to N medical records (as shown in Table 3) according to the topic number K, the LDA model, and the N medical records.

The processor 110 may execute a screening process to generate a corresponding unique drug combination set for each of the K topics. Specifically, the processor 110 may select one or more important drug combinations according to the probability distribution of all drug combinations (that is, M drug combinations) in the topic vector to generate an important drug combination set corresponding to the topic vector. In an embodiment, the processor 110 may use an elbow method to select a plurality of important drug combinations, starting with the drug combination with the maximum probability, from the topic vector to generate an important drug combination set.

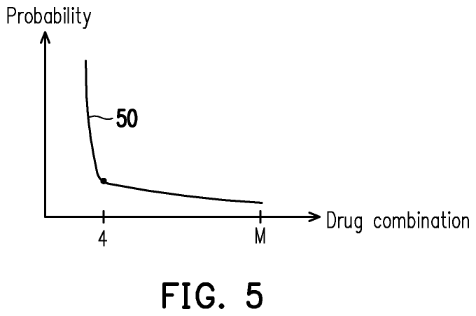

Table 7 is an example of the important drug combination set. Taking topic #1 as an example, the processor 110 may arrange the drug combinations according to the probability according to the topic vector corresponding to topic #1. The drug combinations with greater probabilities are ranked higher. Next, the processor 110 may use the elbow method to find the inflection point of the arranged drug combinations, and select the drug combinations arranged before the inflection point as the important drug combinations. FIG. 5 is a schematic diagram showing finding important drug combinations using the elbow method according to an embodiment of the disclosure. The processor 110 may draw a curve 50 by arranging the drug combinations in a specific topic (for example, topic #1) according to the probability. If the inflection point is the fourth drug combination (that is, the drug combination with the fourth maximum probability), the processor 110 may select the top four drug combinations (that is, the drug combinations with the top four probabilities) from the M drug combinations to generate an important drug combination set. In the topic vector of topic #1, the drug combination (A, B) has the maximum probability, the drug combination (C, D) has the second maximum probability, the drug combination (E, F) has the third maximum probability, and the drug combination (G, H) has the fourth maximum probability. If the drug combination (G, H) corresponds to the inflection point, the processor 110 may select the drug combinations arranged before the drug combination (G, H) to generate the important drug combination set.

TABLE 7

| Topic | Important drug combination set |
|---|---|
| #1 | (A, B), (C, D), (E, F), (G, H) |
| #2 | (G, H), (I, J), (X, Y), (S; W) |
| . . . | . . . |
| #K | (S; W), (O, P), (Q, R) |

After obtaining K important drug combination sets respectively corresponding to the K topics, the processor 110 may generate K unique drug combination sets respectively corresponding to the K topics according to the K important drug combination sets. Specifically, the processor 110 may delete drug combinations that appear repeatedly in different important drug combination sets to generate a unique drug combination set. Taking Table 7 as an example, the processor 110 may delete the drug combination (G, H) from the important drug combination set of topic #1 in response to the drug combination (G, H) being included in the important drug combination set corresponding to topic #1 and being included in the important drug combination set corresponding to topic #2, and thereby generate the unique drug combination set, as shown in Table 8. Table 8 may be the result generated by the processor 110 executing the first screening process.

TABLE 8

| Topic | Unique drug combination set |
|---|---|
| #1 | (A, B), (C, D), (E, F), [symbol] |
| #2 | [symbol] (I, J), (X, Y), [symbol] |
| . . . | . . . |
| #K | [symbol] (O, P), (Q, R) |

Due to the probabilistic nature of the LDA algorithm, the important and unique drug combinations selected in the above-mentioned screening process might appear in this screening process by chance. In order to ensure the stability of the selected drug combinations, the processor 110 may repeatedly execute the screening process multiple times. Specifically, the processor 110 may execute the screening process multiple times to generate a plurality of unique drug combination sets. The processor 110 may generate a stable drug combination set according to specific drug combinations in response to the number of the specific drug combinations in a plurality of unique drug combination sets being greater than a number threshold, wherein the stable drug combination set and the specific drug combinations correspond to the same topic.

Taking the drug combination (A, B) in the unique drug combination set corresponding to topic #1 as an example, it is assumed that the processor 110 executes the screening process 10 times and the number threshold is “6.” If the screening processes in which drug combination (A, B) appears are as shown in Table 9, the processor 110 may determine that the drug combination (A, B) is stable in response to the number of times (that is, 7 times) that the drug combination (A, B) appears in the 10 screening processes being greater than the number threshold. Accordingly, the processor 110 may generate a stable drug combination set corresponding to topic #1 according to the drug combination (A, B), wherein the stable drug combination set corresponding to topic #1 is, for example, the drug combinations (A, B), (C, D), and (E, F) in Table 8.

TABLE 9

| Screen- | | | | | | | | | | |
|---|---|---|---|---|---|---|---|---|---|---|
| ing | | | | | | | | | | |
| process | #1 | #2 | #3 | #4 | #5 | #6 | #7 | #8 | #9 | #10 |
| (A, B) | Yes | Yes | Yes | No | Yes | No | Yes | Yes | No | Yes |
| exists? | | | | | | | | | | |

Table 10 is an example of the stable drug combinations of each topic. After generating K stable drug combination sets respectively corresponding to K topics, the processor 110 may verify whether each stable drug combination set conforms to the drug pattern for enough people. Specifically, the processor 110 may determine the topic corresponding to the medical records according to the maximum probability in the probability distribution of the K topics in the medical record vector. If the maximum probability in the medical record vector of the medical records corresponds to a specific topic, the processor 110 may determine that the medical records correspond to the specific topic. If the medical record vector includes a plurality of maximum probabilities, the processor 110 may determine that the topic corresponding to one of the plurality of maximum probabilities corresponds to the medical records according to a preset rule or randomly. After the determination is made, each topic may correspond to a medical record set including at least one medical record. For example, if a total of 10 medical records among the N medical records correspond to topic #1, it means that the medical record set corresponding to topic #1 includes 10 medical records.

TABLE 10

| Topic | Stable drug combination set |
|---|---|
| #1 | (A, B), (C, D), (E, F) |
| #2 | (I, J), (X, Y) |
| . . . | . . . |
| #K | (O, P), (Q, R) |

The processor 110 may calculate the ratio of at least one medical record in the medical record set to the medical record set, wherein the at least one medical record indicates at least one drug combination in the stable drug combination set. If the ratio is greater than a ratio threshold, it means that the number of samples of medical records conforming to the topic (that is, drug pattern) corresponding to the stable drug combination set is enough. Accordingly, the processor 110 may generate a final drug combination set according to the stable drug combination set. For example, it is assumed that the ratio threshold is 50%. If 60% of the N medical records include at least one drug combination in the stable drug combination set corresponding to topic #1, it means that the samples (that is, medical records) conforming to the drug pattern of topic #1 are enough. Therefore, the processor 110 may generate a final drug combination set according to the stable drug combination set corresponding to topic #1. Table 11 is an example of the medical record set corresponding to topic #1. Relatively speaking, if only 40% of the N medical records include at least one drug combination in the stable drug combination set corresponding to topic #1, it means that the samples conforming to the drug pattern of topic #1 are not enough. Therefore, the processor 110 may not generate the final drug combination set according to the stable drug combination set corresponding to topic #1.

It is assumed that the medical record set corresponding to topic #1 includes at least medical records #10, #11, and #12 (that is, the biased topic of medical records #10, #11, and #12 is topic #1). Referring to Table 10 and Table 11, since the drug combination (C, D) recorded in medical record #10 appears in the stable drug combination set of topic #1, the processor 110 may determine that medical record #10 is one of the above at least one medical record. Since the drug combination (E, F) recorded in medical record #11 appears in the stable drug combination set of topic #1, the processor 110 may determine that medical record #11 is one of the above at least one medical record. Since the drug combination (X, Y) recorded in medical record #12 does not appear in the stable drug combination set of topic #1, the processor 110 may determine that medical record #12 is not one of the above at least one medical record.

TABLE 11
Topic #1

| | | | | | |
|---|---|---|---|---|---|
| Medical | . . . | #10 | #11 | #12 | . . . |
| record | | | | | |
| Drug | . . . | (C, D) | (E, F) | (X, Y) | . . . |
| combination | | | | | |

The processor 110 may check whether each of K stable drug combination sets conforms to the drug pattern for enough people according to the above step. If the stable drug combination set conforms to the drug pattern for enough people, the processor 110 may retain the stable drug combination set. If the stable drug combination set does not conform to the drug pattern for enough people, the processor 110 may delete the stable drug combination set. Accordingly, the processor 110 may select k stable drug combination sets from the K stable drug combination sets respectively corresponding to K topics, wherein k is a positive integer less than or equal to K. The processor 110 may obtain a union of the k stable drug combination sets to obtain a final drug combination set, wherein the drug combination set may include a plurality of drug combinations. Each drug combination in the final drug combination set has the characteristics of high importance, high uniqueness, and high stability, and conforms to the drug pattern of a large number of patients.

After obtaining the final drug combination set, the processor 110 may mark a risk level for the drug combinations in the drug combination set. Specifically, the processor 110 may calculate an odds ratio (OR) for a specific drug combination according to N medical records, as shown in Equation (1) and the confusion matrix in Table 12, wherein e₁y₁ represents the number of medical records that have taken a drug combination and had a hospitalization event in the N medical records, e₁y₀ represents the number of medical records that have taken a drug combination but did not have a hospitalization event in the N medical records, e₀y₁ represents the number of medical records that did not take a drug combination but have had a hospitalization event in the N medical records, and e₀y₀ represents the number of medical records that did not take a drug combination and did not have a hospitalization event.

$$OR = \frac{{e}_{1} {y}_{1} \times {e}_{0} {y}_{0}}{{e}_{0} {y}_{1} \times {e}_{1} {y}_{0}} \qquad ( 1 )$$

TABLE 12

| | Had a | Did not have a |
|---|---|---|
| | hospitalization | hospitalization |
| N medical records | event | event |
| Took the drug | e₁y₁ | e₁y₀ |
| combination | | |
| Did not take the | e₀y₁ | e₀y₀ |
| drug combination | | |

After calculating the odds ratio of each drug combination in the drug combination set, the processor 110 may mark the risk level for the drug combination according to the odds ratio of the drug combination. If the odds ratio of the drug combination is greater than a risk threshold, it means that the drug combination is very likely to be the cause of the hospitalization event. Accordingly, the processor 110 may mark the drug combination as high risk. In contrast, if the odds ratio of the drug combination is less than or equal to the risk threshold, it means that the drug combination is less associated with the occurrence of the hospitalization event. Accordingly, the processor 110 may mark the drug combination as low risk. Table 13 is an example of the risk levels marked for drug combinations. Assuming that the risk threshold is “1.3,” the processor 110 may mark drug combinations with an odds ratio greater than “1.3” as high risk, and mark drug combinations with an odds ratio less than or equal to “1.3” as low risk.

TABLE 13

| Drug | | |
|---|---|---|
| combination | OR | Mark |
| (A, C) | 1.5 | High risk |
| (A, D) | 0.9 | Low risk |
| (A, E) | 1.1 | Low risk |
| (A, F) | 2.7 | High risk |
| (B, G) | 2.7 | High risk |
| (B, H) | 0.8 | Low risk |
| (B, I) | 6.1 | High risk |
| (B, J) | 3.0 | High risk |

After marking each drug combination in the drug combination set, the processor 110 may generate a risk combination fraction (RCF) corresponding to a drug in the drug combination according to the marked drug combination. Taking the drug combination (α, β) as an example, in order to confirm that the combination of drug α in the drug combination (α, β) and other drugs in the drug combination set (that is, drugs other than drug β) is safe, the processor 110 may calculate the risk combination fraction RCF (or referred to as “third fraction”) corresponding to drug α according to Equation (2), wherein S(α, β) is the number of drug combinations that include drug α but do not include drug β in the drug combination set, and S′(α, β) is the number of drug combinations that include drug α but do not include drug β and are marked as high risk in the drug combination set.

$$RCF = \frac{S ′ ( α , β )}{S ( α , β )} \qquad ( 2 )$$

After obtaining the RCF of drug α, the processor 110 may calculate a normal combination fraction (NCF) (or referred to as “first fraction” or “second fraction”) of drug α according to Equation (3). A higher normal combination fraction of drug α represents a lower risk of drug α being combined with other drugs other than drug β. The NCF of drug α may be negatively correlated with the odds ratio of the drug combination including drug α but not including drug β. Taking Table 13 as an example, the NCF of drug A may be negatively correlated with the odds ratio of the drug combination (A, C), (A, D), (A, E) or (A, F).

NCF=1−RCF (3)

Taking drug A in Table 13 as an example, it is assumed that Table 13 includes all drug combinations except for the drug combination (A, B) in the drug combination set, wherein there are two drug combinations that include drug A and are marked as high risk, which are drug combinations (A, C) and (A, F). Accordingly, the processor 110 may calculate to obtain that the RCF of drug A is equal to “0.5” and the NCF of drug A is equal to “0.5” according to Equations (2) and (3). Taking drug B in Table 13 as an example, there are three drug combinations that include drug B and are marked as high risk in Table 13, which are drug combinations (B, G), (B, I) and (B, J). Accordingly, the processor 110 may calculate to obtain that the RCF of drug B is equal to “0.75” and the NCF of drug B is equal to “0.25” according to Equations (2) and (3).

Assuming that the NCF of drug α is greater than or equal to the NCF of drug β, after obtaining the NCF of drug α and the NCF of drug β, the processor 110 may calculate a quotient (or ratio) Q(α, β) of the NCF of drug α and the NCF of drug β, as shown in Equation (4), wherein NCF(α) is the NCF of drug α and NCF(β) is the NCF of drug β. The value of Q(α, β) is greater than or equal to 1, and a smaller value represents that the risk of drug α being combined with other drugs other than drug β and the risk of drug β being combined with other drugs other than drug α are closer.

$$Q ( α , β ) = \frac{NCF ( α )}{NCF ( β )} \qquad ( 4 )$$

The processor 110 may determine whether a specific drug combination has a high risk of interaction according to the following three conditions. Taking the drug combination (α, β) as an example, if the odds ratio of the drug combination (α, β) is greater than a first threshold, the sum of NCF(α) of drug α and NCF(β) of drug β (or referred to as NCF sum) is greater than a second threshold, and the quotient of NCF(α) of drug α and NCF(β) of drug β (or referred to as NCF quotient) is less than a third threshold, the processor 110 may determine that the drug combination (α, β) has a high risk of interaction. The processor 110 may output the drug combination (α, β) through the transceiver 130 for the user's reference.

Table 14 shows the odds ratios of multiple drug combinations and related parameters of NCF. It is assumed that the first threshold is “2.0,” the second threshold is “1.2,” and the third threshold is “1.8.” Since the odds ratio of the drug combination (E, F) is greater than the first threshold, the NCF sum is greater than the second threshold, and the NCF quotient is less than the third threshold, the processor 110 may determine that the drug combination (E, F) fully meets the three conditions. Accordingly, the processor 110 may output the drug combination (E, F). Since the odds ratio of the drug combination (G, H) is less than the first threshold, the processor 110 may determine that the drug combination (G, H) does not fully meet the three conditions. Accordingly, the processor 110 may not output the drug combination (G, H). Since the NCF sum of the drug combination (A, B) is less than the second threshold or the NCF quotient is greater than the third threshold, the processor 110 may determine that the drug combination (A, B) does not fully meet the three conditions. Accordingly, the processor 110 may not output the drug combination (A, B). Since the NCF quotient of the drug combination (C, D) is greater than the third threshold, the processor 110 may determine that the drug combination (C, D) does not fully meet the three conditions. Accordingly, the processor 110 may not output the drug combination (C, D).

TABLE 14

| Drug | | | | | |
|---|---|---|---|---|---|
| combination | | NCF | NCF | NCF | NCF |
| (α, β) | OR | (α, other) | (ß, other) | sum | quotient |
| (E, F) | 2.38 | 0.695 | 0.652 | 1.347 | 1.065 |
| (G, H) | 1.02 | 0.714 | 0.640 | 1.354 | 1.116 |
| (A, B) | 8.53 | 0.500 | 0.250 | 0.750 | 2.000 |
| (C, D) | 2.18 | 1.001 | 0.500 | 1.501 | 2.002 |

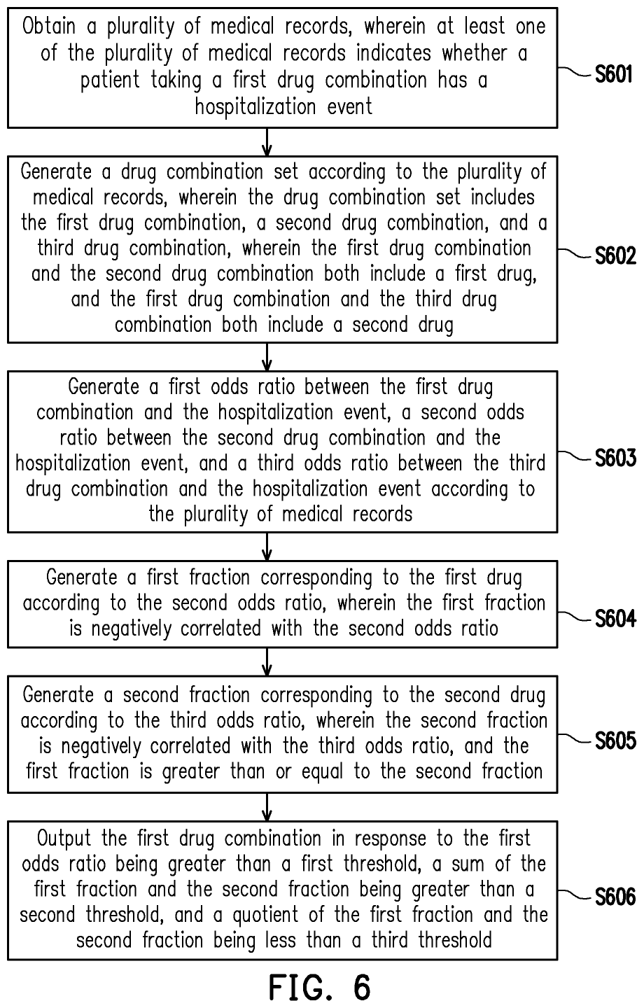

FIG. 6 is a flowchart showing a method of checking drug interaction according to an embodiment of the disclosure, and the method can be implemented by the electronic device 100 as shown in FIG. 1. In step S601, a plurality of medical records are obtained, wherein at least one of the plurality of medical records indicates whether a patient taking a first drug combination has a hospitalization event. In step S602, a drug combination set is generated according to the plurality of medical records, wherein the drug combination set includes the first drug combination, a second drug combination, and a third drug combination. The first drug combination and the second drug combination both include a first drug, and the first drug combination and the third drug combination both include a second drug. In step S603, a first odds ratio between the first drug combination and the hospitalization event, a second odds ratio between the second drug combination and the hospitalization event, and a third odds ratio between the third drug combination and the hospitalization event are generated according to the plurality of medical records. In step S604, a first fraction corresponding to the first drug is generated according to the second odds ratio, wherein the first fraction is negatively correlated with the second odds ratio. In step S605, a second fraction corresponding to the second drug is generated according to the third odds ratio, wherein the second fraction is negatively correlated with the third odds ratio, and the first fraction is greater than or equal to the second fraction. In step S606, in response to the first odds ratio being greater than a first threshold, a sum of the first fraction and the second fraction being greater than a second threshold, and a quotient of the first fraction and the second fraction being less than a third threshold, the first drug combination is output.

To sum up, the electronic device according to the disclosure can analyze multiple medical records through the latent Dirichlet allocation model, and select the optimal number of for the drug combination group based on factors such as the similarity of the drug pattern, the biased drug pattern of the patient or the clustering efficiency index. After assigning each drug combination group to a specific drug pattern according to the optimal number, the electronic device can select the most representative drug combinations according to factors such as the importance, uniqueness, stability, and number of samples of the drug combinations. If two safe drugs in a specific drug combination are likely to cause adverse interaction, the information of the specific drug combination is output for the user's reference.

What is claimed is:

1. A method of checking drug interaction, comprising:
obtaining a plurality of medical records, wherein at least one of the plurality of medical records indicates whether a patient taking a first drug combination has a hospitalization event;
generating a drug combination set according to the plurality of medical records, wherein the drug combination set comprises the first drug combination, a second drug combination, and a third drug combination, wherein the first drug combination and the second drug combination both comprise a first drug, and the first drug combination and the third drug combination both comprise a second drug;
generating a first odds ratio between the first drug combination and the hospitalization event, a second odds ratio between the second drug combination and the hospitalization event, and a third odds ratio between the third drug combination and the hospitalization event according to the plurality of medical records;
generating a first fraction corresponding to the first drug according to the second odds ratio, wherein the first fraction is negatively correlated with the second odds ratio;
generating a second fraction corresponding to the second drug according to the third odds ratio, wherein the second fraction is negatively correlated with the third odds ratio, and the first fraction is greater than or equal to the second fraction; and
outputting the first drug combination in response to the first odds ratio being greater than a first threshold, a sum of the first fraction and the second fraction being greater than a second threshold, and a quotient of the first fraction and the second fraction being less than a third threshold.

2. The method of checking drug interaction according to claim 1, wherein generating the first fraction corresponding to the first drug according to the second odds ratio comprises:
marking the second drug combination in response to the second odds ratio being greater than a risk threshold;
generating a third fraction according to the second drug combination marked, wherein the third fraction is equal to the number of drug combinations comprising the first drug but not comprising the second drug and being marked in the drug combination set, divided by the number of drug combinations comprising the first drug but not comprising the second drug in the drug combination set; and
calculating the first fraction according to the third fraction, wherein a sum of the first fraction and the third fraction is equal to 1.

3. The method of checking drug interaction according to claim 1, wherein generating the drug combination set according to the plurality of medical records comprises:
executing a screening process to generate a first unique drug combination set, comprising:
generating K topic vectors comprising a first topic vector according to the plurality of medical records and a latent Dirichlet allocation model, wherein K is a first topic number, the K topic vectors respectively correspond to K topics, the K topics comprise a first topic corresponding to the first topic vector, and the first topic vector comprises a probability distribution of all drug combinations;
selecting a plurality of important drug combinations, starting with a drug combination with a maximum probability, from the first topic vector to generate a first important drug combination set; and
determining the first unique drug combination set according to the first important drug combination set; and
generating the drug combination set according to the first unique drug combination set.

4. The method of checking drug interaction according to claim 3, wherein the K topics comprise a second topic, and determining the first unique drug combination set according to the first important drug combination set comprises:
deleting a first important drug combination from the first important drug combination set to generate the first unique drug combination set in response to the first important drug combination being included in the first important drug combination set corresponding to the first topic and a second important drug combination set corresponding to the second topic.

5. The method of checking drug interaction according to claim 3, wherein generating the drug combination set according to the first unique drug combination set comprises:
repeatedly executing the screening process multiple times to generate a plurality of unique drug combination sets comprising the first unique drug combination set;
generating a first stable drug combination set corresponding to the first topic according to the first drug combination in response to the number of the first drug combinations in the plurality of unique drug combination sets being greater than a number threshold; and
generating the drug combination set according to the first stable drug combination set.

6. The method of checking drug interaction according to claim 5, wherein generating the drug combination set according to the first stable drug combination set comprises:
generating a plurality of medical record vectors respectively corresponding to the plurality of medical records according to the plurality of medical records and the latent Dirichlet allocation model, wherein each of the plurality of medical record vectors comprises a probability distribution of the K topics;
determining a medical record set corresponding to the first topic among the plurality of medical records according to the probability distribution of the K topics;
calculating a ratio of at least one medical record in the medical record set to the medical record set, wherein the at least one medical record indicates at least one drug combination in the first stable drug combination set; and
generating the drug combination set according to the first stable drug combination set in response to the ratio being greater than a ratio threshold, wherein the drug combination set comprises a plurality of drug combinations in the first stable drug combination set.

7. The method of checking drug interaction according to claim 6, wherein a first medical record in the medical record set corresponds to a first probability distribution of the K topics, and determining the medical record set corresponding to the first topic among the plurality of medical records according to the probability distribution of the K topics comprises:
determining that the first medical record corresponds to the first topic in response to a maximum probability in the first probability distribution corresponding to the first topic.

8. The method of checking drug interaction according to claim 3, further comprising:
generating a first index corresponding to the first topic number and a second index corresponding to a second topic number according to the plurality of medical records and the latent Dirichlet allocation model; and
comparing the first index and the second index to select the first topic number from the first topic number and the second topic number as K.

9. The method of checking drug interaction according to claim 8, wherein generating the first index corresponding to the first topic number comprises:
generating the K topic vectors according to the plurality of medical records, the latent Dirichlet allocation model, and the first topic number; and
calculating an average similarity of all 2-combinations of the K topic vectors as the first index.

10. The method of checking drug interaction according to claim 8, wherein generating the first index corresponding to the first topic number comprises:
generating a plurality of medical record vectors respectively corresponding to the plurality of medical records according to the plurality of medical records, the latent Dirichlet allocation model, and the first topic number, wherein each of the plurality of medical record vectors comprises a probability distribution of the K topics;
determining at least one medical record corresponding to the first topic among the plurality of medical records according to the probability distribution of the K topics; and
calculating a ratio according to the number of the at least one medical record and a total number of the plurality of medical records as the first index.

11. The method of checking drug interaction according to claim 10, wherein determining the at least one medical record corresponding to the first topic among the plurality of medical records according to the probability distribution of the K topics comprises:
obtaining a first probability distribution corresponding to the K topics of the at least one medical record from the plurality of medical record vectors; and
determining that the at least one medical record corresponds to the first topic in response to a maximum probability in the first probability distribution corresponding to the first topic and being greater than a probability threshold.

12. The method of checking drug interaction according to claim 8, wherein generating the first index corresponding to the first topic number comprises:
generating a plurality of medical record vectors respectively corresponding to the plurality of medical records according to the plurality of medical records, the latent Dirichlet allocation model, and the first topic number, wherein each of the plurality of medical record vectors comprises a probability distribution of the K topics;
dividing the plurality of medical records into K groups according to the probability distribution of the K topics, wherein the K groups respectively correspond to the K topics;
calculating a first statistical value of inter-group distances according to the K groups;
calculating a second statistical value of intra-group distances according to the K groups; and
calculating a ratio of the first statistical value to the second statistical value as the first index.

13. The method of checking drug interaction according to claim 12, wherein calculating the first statistical value of the inter-group distances according to the K groups comprises:
calculating a plurality of distances between the K topic vectors; and
obtaining the first statistical value by adding the plurality of distances.

14. The method of checking drug interaction according to claim 12, wherein the K groups comprise a first group and a second group, and calculating the second statistical value of the intra-group distances according to the K groups comprises:
calculating a plurality of distances between a plurality of elements in the first group to generate a first sum of intra-group distances corresponding to the first group; and
obtaining the second statistical value by adding the first sum of the intra-group distances corresponding to the first group and a second sum of intra-group distances corresponding to the second group.

15. An electronic device of checking drug interaction, comprising:
a transceiver; and
a processor coupled to the transceiver and configured to:
obtain, through the transceiver, a plurality of medical records, wherein at least one of the plurality of medical records indicates whether a patient taking a first drug combination has a hospitalization event;
generate a drug combination set according to the plurality of medical records, wherein the drug combination set comprises the first drug combination, a second drug combination, and a third drug combination, wherein the first drug combination and the second drug combination both comprise a first drug, and the first drug combination and the third drug combination both comprise a second drug;
generate a first odds ratio between the first drug combination and the hospitalization event, a second odds ratio between the second drug combination and the hospitalization event, and a third odds ratio between the third drug combination and the hospitalization event according to the plurality of medical records;
generate a first fraction corresponding to the first drug according to the second odds ratio, wherein the first fraction is negatively correlated with the second odds ratio;
generate a second fraction corresponding to the second drug according to the third odds ratio, wherein the second fraction is negatively correlated with the third odds ratio, and the first fraction is greater than or equal to the second fraction; and
output, through the transceiver, the first drug combination in response to the first odds ratio being greater than a first threshold, a sum of the first fraction and the second fraction being greater than a second threshold, and a quotient of the first fraction and the second fraction being less than a third threshold.
